(19) United States
(12) Patent Application Publication
(10) Pub. No.: US 2009/0187056 A1
(43) Pub. Date: Jul. 23, 2009

(54) PROCESS FOR THE PREPARATION OF AN OLEFIN

(76) Inventors: Leslie Andrew Chewter, Amsterdam (NL); Michiel Johannes Franciscus Maria Verhaak, Amsterdam (NL); Jeroen Van Westrenen, Amsterdam (NL)

Correspondence Address: SHELL OIL COMPANY, P O BOX 2463, HOUSTON, TX, 772522463, US

(21) Appl. No.: 12/301,120

(22) PCT Filed: May 16, 2007
(86) PCT No.: PCT/EP2007/054758

§ 371 Date: Dec. 9, 2008

(30) Foreign Application Priority Data
May 19, 2006 (EP) 06114289.9

(51) Int. Cl. C07C 2/86

(52) U.S. Cl. 585/638; 585/639

(57) ABSTRACT

Process for the preparation of an olefin comprising contacting a diluted olefinic hydrocarbon feed, containing an oxygenate feed, an olefinic co-feed and one or more diluents, with a solid zeolite catalyst to obtain a reaction product containing one or more olefins, and wherein the diluted feed is contacted with the solid zeolite catalyst at a Gas Hourly Space Velocity, as measured at standard temperature and pressure, of at least 10,000 ml diluted olefinic hydrocarbon feed/gram zeolite catalyst/hour.

### FIELD OF THE INVENTION

This invention relates to a process for the preparation of an olefin. In specific this invention relates to a process for the conversion of oxygenates into olefins.

### BACKGROUND OF THE INVENTION

Processes for the preparation of olefins are known in the art.

EP-A-0485145 describes a process for the production of olefins which comprises passing an oxygenate-containing feedstock over a zeo type catalyst at a temperature greater than 200° C., where the catalyst containing feedstock comprises C3 and/or C4 olefins and as oxygenate methanol, formaldehyde and/or dimethylether is used. The process is directed to the selective production of C4/C5 olefins. In example 4 a gaseous feed of 1-butene, methanol and nitrogen was led over a Theta-1 zeolite catalyst. A total of 5551 mmol, respectively 5591 mmol gas was fed per hour over the catalyst at 400° C. The Gas Hourly Space Velocity at standard conditions of 23° C. and 1 bar can be calculated to be respectively 8,987 and 9,052 ml/gram zeolite/hour.

US2003/0078463 in the name of Exxon Mobile describes a process for making ethene and propene from an oxygenate feed using two or more zeolite catalysts.

In a first stage, oxygenate contacts a zeolite catalyst, preferably containing ZSM-5. The resulting oxygenate conversion product contains ethylene, propylene, and butenes and higher olefin composition. The olefin product from the oxygenate conversion reaction, with or without prior separation of ethylene and propylene, then contacts another zeolite catalyst in a second stage. The catalyst is such second stage is a 10-ring zeolite, including ZSM-22, ZSM-23, ZSM-35, ZSM-42 or mixtures thereof.

In passing, it is described that hydrocarbons can also be included as part of the feedstock in the first stage, i.e. as co-feed. It is described that desirably, the oxygenate feedstock can be contacted with the catalyst at a Weight Hourly Space Velocity (WHSV) in the range from about 1 hr-1 to about 1000 hr-1, preferably from about 20 hr-1 to about 500 hr-1.

In the only example, pure methanol is converted by a two-step process into several olefins. The WHSV is not indicated.

It would be desirable to have an improved process, in which there is a flexibility towards either higher olefins, such as pentene and/or hexene, or lower olefins such as ethylene and/or propylene.

### SUMMARY OF THE INVENTION

Such an improved process can be obtained by using a diluted feed at a high Gas Hourly Space Velocity.

Accordingly, the present invention provides a process for the preparation of an olefin comprising contacting a diluted olefinic hydrocarbon feed, containing an oxygenate feed, an olefinic co-feed and one or more diluents, with a solid zeolite catalyst to obtain a reaction product containing one or more olefins, and wherein the diluted feed is contacted with the solid zeolite catalyst at a Gas Hourly Space Velocity, as measured at standard temperature and pressure of 23° C. and 1 bar, of at least 10,000 ml diluted olefinic hydrocarbon feed/gram zeolite catalyst/hour.

As shown in the examples, the process according to the invention allows flexibility towards either higher olefins, such as pentene and/or hexene, or lower olefins such as ethylene and/or propylene. Moreover, such flexibility is allowed within a one-step process. Advantageously with the process according to the invention a high selectivity towards ethylene and/or propylene can be obtained. By using slightly different GHSV's a high selectivity towards pentenes and/or hexenes can be obtained.

### DETAILED DESCRIPTION OF THE INVENTION

By a diluted olefinic hydrocarbon feed is understood a feed containing one or more olefins, one or more oxygenates and one or more diluents. Such feed may herein also be referred to as diluted feed.

By an olefinic co-feed is understood a feed containing one or more olefins. The olefinic co-feed can contain one olefin or a mixture of olefins. Preferably the olefinic co-feed contains a mixture of olefins. Apart from olefins, the olefinic co-feed may contain other hydrocarbon compounds, such as for example paraffinic, alkylaromatic, aromatic compounds or a mixture thereof. Preferably the olefinic co-feed comprises more than 50 wt %, more preferably more than 80 wt %, still more preferably more than 90 wt % and most preferably in the range from 95 to 100 wt % of olefin(s). An especially preferred olefinic co-feed consists essentially of olefin(s).

Any non-olefinic compounds in the olefinic co-feed are preferably paraffinic compounds. If the olefinic co-feed contains any non-olefinic hydrocarbon, these are preferably paraffinic compounds. Such paraffinic compounds are preferably present in an amount in the range from 0 to 10 wt %, more preferably in the range from 0 to 5 wt %, still more preferably in the range from 0 to 1 wt % and most preferably in an amount of less than 0.5 wt %.

By an olefin is understood an organic compound containing at least two carbon atoms connected by a double bond. A wide range of olefins can be used. The olefin can be a mono-olefin, having one double bond, or a poly-olefin, having two or more double bonds. Preferably olefins present in the olefinic co-feed are mono-olefins.

The olefin(s) can be a linear, branched or cyclic olefin. Preferably olefins present in the olefinic co-feed are linear or branched olefins.

Preferred olefins have in the range from 2 to 12, preferably in the range from 3 to 10, and more preferably in the range from 4 to 8 carbon atoms.

Examples of suitable olefins that may be contained in the olefinic co-feed include ethene, propene, 1-butene, 2-butene, iso-butene (2-methyl-1-propene), 1-pentene, 2-pentene, 2-methyl-1-butene, 2-methyl-2-butene, 3-methyl-1-butene, 1-hexene, 2-hexene, 3-hexene, 2-methyl-1-pentene, 2-methyl-2-pentene, 3-methyl-1-pentene, 3-methyl-2-pentene, 4-methyl-1-pentene, 4-methyl-2-pentene, 2,3-dimethyl-1-butene, 2,3-dimethyl-2-butene, 3,3-dimethyl-1-butene, cyclopentene, methylcyclopentene or cyclohexene, heptenes, octenes, nonenes and decenes. The preference for specific olefins in the olefinic co-feed may depend on the purpose of the process.

In one embodiment, where the purpose of the process is to prepare ethene and/or propene, the olefinic co-feed preferably contains olefins having 4 or more carbon atoms (i.e. C₄₊ olefins), such as butenes, pentenes, hexenes and heptenes.

In another embodiment, where the purpose of the process is to prepare pentenes and/or hexenes, the olefinic co-feed preferably contains olefins having 4 or less carbon atoms (i.e. C₄₋ olefins), such as butenes, propene and ethene. In a still further embodiment, where the purpose of the process is to prepare ethene, propene, pentene and/or hexene, the olefinic co-feed preferably contains only olefins having 4 carbon atoms.

Of the above embodiments, the first embodiment wherein ethene and propene is prepared and the olefinic co-feed contains olefins having 4 or more carbon atoms (i.e. C₄₊ olefins), such as butenes, pentenes, hexenes and heptenes, is preferred.

Preferably reaction product containing one or more olefins is separated into one or more fractions, preferably at least a product fraction containing olefins intended as a product and a further fraction. Preferably at least part of the further fraction is recycled to the start of the process as (part of) the olefinic co-feed.

The olefinic co-feed preferably consists for at least 50 wt %, more preferably at least 80 wt %, still more preferably from 90 to 100 wt % from such recycled olefins from the reaction product. In a specifically preferred embodiment the olefinic co-feed consists essentially of recycled olefins from the reaction product.

By an oxygenate feed is understood a feed comprising an oxygenate.

By an oxygenate is understood a compound obtainable by oxidation of a hydrocarbon. Water is therefore not understood to be an oxygenate. Examples of oxygenates include alcohols, such as methanol, ethanol, isopropanol, ethylene glycol, propylene glycol; ketones, such as acetone and methylethylketone; aldehydes, such as formaldehyde, acetaldehyde and propionaldehyde; ethers, such as dimethylether, diethylether, methylethylether, tetrahydrofuran and dioxane; epoxides such as ethylene oxide and propylene oxide; and acids, such as acetic acid, propionic acid, formic acid and butyric acid. Of these alcohols and ethers are preferred.

The oxygenate used in the process according to the invention is preferably an oxygenate which comprises at least one oxygen-bound C1-C4 alkyl group. The oxygenate can comprise one or more oxygen-bound C1-C4 alkyl group. Preferably the oxygenate comprises one or two C1-C4 alkyl groups. Preferably the oxygenate is chosen from the group of dimethylether, diethylether, methylethylether, methanol, ethanol and isopropanol.

More preferably an oxygenate is used having at least one oxygen-bonded C₁ or C₂ alkyl group, still more preferably at least one oxygen-bonded C₁ group. Most preferably the oxygenate is methanol or dimethylether.

In a preferred embodiment, where the oxygenate is methanol, such methanol is obtained from natural gas. For example by a process as described in Industrial Organic Chemistry 3rd edition page 28.

In another preferred embodiment the oxygenate is obtained through fermentation of biomaterials. For example by a process as described DE-A-10043644.

The preferred molar ratio of oxygenate to olefin in the olefinic co-feed depends on the specific oxygenate used and the number of reactive oxygen-bonded alkyl groups therein. Preferably the ratio of mol oxygenate to mol olefin lies in the range of 10:1 to 1:10, more preferably in the range of 5:1 to 1:5 and still more preferably in the range of 2:1 to 1:2.

In a preferred embodiment wherein the oxygenate comprises only one oxygen-bonded alkyl group, such as for example methanol or ethanol, the molar ratio preferably lies in the range from 5:1 to 1:5 and more preferably in the range of 2:1 to 1:2. Most preferably the molar ratio in such a case is about 1:1.

In another preferred embodiment wherein the oxygenate comprises two oxygen-bonded alkyl group, such as for example dimethylether, the molar ratio preferably lies in the range from 5:2 to 1:10 and more preferably in the range of 1:1 to 1:4. Most preferably the molar ratio in such a case is about 1:2.

The diluted olefinic hydrocarbon feed contains one or more olefins, one or more oxygenate and one or more diluents. Preferably the diluted feed comprises in the range of 1 to 99 vol %, more preferably in the range of 10 to 98 vol %, still more preferably in the range from 20 to 97 vol % diluent, based on the total volume of diluted feed.

Still more preferably the amount of diluent is at least 50 vol %, based on the total volume of diluted feed. The remainder preferably being oxygenate(s) and olefin(s). Although other components might be present in the diluted feed, the diluted feed preferably consists of only one or more oxygenates, one or more olefins and one or more diluents.

Any diluent known by the skilled person to be suitable for such purpose can be used. Such diluent can for example be a paraffinic compound or mixture of compounds. Preferably, however, the diluent is an inert gas. More preferably, the diluent is chosen from the group of inert gases such as argon, nitrogen and steam. Of these, steam is the most preferred diluent. For example, the oxygenate feed and/or olefinic co-feed can be diluted with steam, for example in the range from 0.01 to 10 kg steam per kg feed.

The diluent can be mixed with the oxygenate feed and/or olefinic co-feed in a separate mixing nozzle, possibly whilst using mixing devices to enhance the mixing process. Or, if desirable, a product from another process containing olefins and a diluent can be used as diluted olefinic hydrocarbon feed.

The diluted feed, containing olefin, oxygenate and diluent, is contacted with a solid zeolite catalyst.

By a zeolite catalyst is understood a catalyst containing a zeolite.

Preferably, the zeolite is a zeolite comprising a 10-membered ring channel. More preferably this zeolite is a one-dimensional zeolite having 10-membered ring channels. A one-dimensional zeolite having 10-membered ring channels is understood to be a zeolite having only 10-membered ring channels in one direction which are not intersected by other 8, 10 or 12-membered ring channels from another direction.

One suitable zeolite is a zeolite of the MFI-type (for example ZSM-5). Preferably, however, the zeolite is selected from the group of TON-type (for example ZSM-22), MTT-type (for example ZSM-23), STF-type (for example SSZ-35), SFF-type (for example SSZ-44) and EU-2-type/ ZSM-48 zeolites.

The preferred zeolites used in the present invention are distinct from zeolites having small pore 8-ring channels or zeolites having large pore 12-ring channels.

MTT-type catalysts are more particularly described in e.g. U.S. Pat. No. 4,076,842. For purposes of the present invention, MTT is considered to include its isotypes, e.g., ZSM-23, EU-13, ISI-4 and KZ-1.

TON-type zeolites are more particularly described in e.g. U.S. Pat. No. 4,556,477. For purposes of the present invention, TON is considered to include its isotypes, e.g., ZSM-22, Theta-1, ISI-1, KZ-2 and NU-10.

EU-2-type zeolites are more particularly described in e.g. U.S. Pat. No. 4,397,827. For purposes of the present invention, EU-2 is considered to include its isotypes, e.g., ZSM-48.

In a further preferred embodiment a zeolite of the MTT-type, such as ZSM-23, is used.

Preferably a zeolite in the hydrogen form is used, e.g., HZSM-22, HZSM-23, HZSM-48. Preferably at least 50% w/w, more preferably at least 90% w/w, still more preferably at least 95% w/w and most preferably 100% of the total amount of zeolite used is zeolite in the hydrogen form. When the zeolites are prepared in the presence of organic cations the zeolite may be activated by heating in an inert or oxidative atmosphere to remove the organic cations, for example, by heating at a temperature over 500° C. for 1 hour or more. The hydrogen form can then be obtained by an ion exchange procedure with ammonium salts followed by another heat treatment, for example in an inert or oxidative atmosphere at a temperature over 500° C. for 1 hour or more. The zeolites obtained after ion exchange with ammonium salts are also referred to as being in the ammonium form.

Preferably the zeolite has a silica to alumina ratio (SAR) in the range from 1 to 500. More preferably the zeolite has a SAR in the range from 10 to 200, still more preferably the zeolite has a SAR in the range from 10 to 100.

The zeolite can be used as such or in combination with a so-called binder material. If no binder material is used the zeolite is referred to as zeolite catalyst. If a binder is used the zeolite in combination with the binder material is referred to as zeolite catalyst.

It is desirable to provide a zeolite catalyst having good mechanical strength, because in an industrial environment the catalyst is often subjected to rough handling which tends to break down the catalyst into powder-like material. The later causes problems in the processing. Preferably the zeolite is therefore incorporated in a binder material. Examples of suitable binder materials include active and inactive materials and synthetic or naturally occurring zeolites as well as inorganic materials such as clays, silica, alumina, aluminosilicate. For present purposes, inactive materials of a low acidity, such as silica, are preferred because they may prevent unwanted side reactions which may take place in case a more acidic material, such as alumina is used. Preferably the catalyst used in the process of the present invention comprises, in addition to the zeolite, 2 to 90 wt %, preferably 10 to 85 wt % of a binder material.

The process of the present invention can be carried out in a batch, continuous, semi-batch or semi-continuous manner. Preferably the process of the present invention is carried out in a continuous manner.

If the process is carried out in a continuous manner, the process may be started up by using olefins obtained from an external source for the olefinic co-feed and continued with olefins obtained from recycling part of the reaction product.

Olefins for the start-up may for example be obtained from a steam cracker, a catalytic cracker or alkane dehydrogenation (e.g. propane or butane dehydrogenation). Further, such olefins can be bought from the market.

The reactor used may be any reactor known to the skilled person and may for example contain a fixed bed, moving bed and/or a fluidized bed.

Conventional catalyst regeneration techniques can be employed. The zeolite catalyst used in the process of the present invention can have any shape known to the skilled person to be suitable for this purpose, for it can be present in the form of tablets, rings, extrudates, etc. extruded catalysts can be applied in various shapes, such as, cylinders and trilobes. If desired, spent zeolite can be regenerated and recycled to the process of the invention.

The process can be carried out over a wide range of temperatures and pressures. Preferably, however, the hydrocarbon feed is contacted with the zeolite at a temperature in the range from 200° C. to 600° C. In a further preferred embodiment the temperature is in the range from 250° C. to 550° C., more preferably in the range from 300° C. to 450° C.

In another preferred embodiment the temperature is in the range from 425° C. to 575° C., more preferably in the range from 450° C. to 550° C.
The pressure can vary widely, preferably a pressure in the range from 1 to 5 bar is applied, more preferably a pressure in the range of 1 to 3 bar is applied. The partial pressure of the olefinic co-feed can be calculated multiplying the pressure applied with the vol %, that is if the volume percent is for example 5 vol % than the pressure is multiplied by ( 5/100), i.e. 0.05.

The Gas Hourly Space Velocity (GHSV), as measured at standard temperature and pressure (STP) of 23° C. and 1 bar, for such a process can vary over a wide range, starting from e.g. 2,000 ml/gram zeolite catalyst/hour or 3,000 ml/gram zeolite catalyst/hour. For the process of the invention, however, it has been found advantageous to use a Gas Hourly Space Velocity (GHSV) of at least 10,000 ml diluted olefinic hydrocarbon feed/gram zeolite catalyst/hour under standard conditions (STP) of 23° C. and 1 bar. More preferably such GHSV is at least 12,000 ml/gram zeolite catalyst/hour. Although there is no maximum, an upper limit may be determined by the dimensions of the equipment available. For practical purposes the GHSV is preferably at most 1,000,000 ml/gram zeolite catalyst/hour, more preferably at most 500,000 ml/gram zeolite catalyst/hour. In an especially preferred embodiment the GHSV lies in the range from 10,000 to 40,000 ml/gram zeolite catalyst/hour.

When the purpose of the process is to prepare ethylene and/or propylene, a preferred GHSV range is the range from 10,000 to 60,000 ml/gram zeolite catalyst/hour, more preferably 10,000 to 40,000 ml/gram zeolite catalyst/hour, still more preferably between 10,000 to 25,000 ml/gram zeolite catalyst/hour.

When the purpose of the process is to prepare pentenes and/or hexenes, a preferred GHSV range is the range from 20,000 to 500,000 ml/gram zeolite catalyst/hour, more preferably between 25,000 to 300,000 ml/gram zeolite catalyst/hour, still more preferably between 30,000 to 300,000 ml/gram zeolite catalyst/hour.

The most preferred GHSV depends on the dilution applied. When the concentration of olefinic hydrocarbon feed in the diluted olefinic hydrocarbon feed is at least 30 vol %, a GHSV (STP) in the range from 10,000 to 100,000 ml/gram zeolite catalyst/hour, more preferably 12,000 to 100,000 ml/gram zeolite catalyst/hour, may be preferred. When the concentration of olefinic hydrocarbon feed in the diluted olefinic hydrocarbon feed is at most 30 vol %, a GHSV in the range from 25,000 to 500,000 ml/gram zeolite catalyst/hour, more preferably 50,000 to 500,000 ml/gram zeolite catalyst/hour, may be preferred.

In another advantageous embodiment, a Gas Hourly Space Velocity (GHSV) of at least 10,000 ml, preferably at least 12,000 ml, still more preferably at least 20,000 ml diluted olefinic hydrocarbon feed/gram zeolite/hour under standard conditions (STP) of 23° C. and 1 bar is used. If the catalyst comprises both a zeolite and a binder, such GHSV based on gram zeolite/hour is calculated on the grams of zeolite only. For practical purposes such GHSV based on gram zeolite/hour is preferably at most 1,000,000 ml/gram zeolite/hour, more preferably at most 500,000 ml/gram zeolite/hour.

Gas Hourly Space Velocity is measured at a, within this specification defined as, standard temperature of 23° C. and a standard pressure of 1 bar (STP). By means of the ideal gas law (i.e. pressure times volume divided by temperature is constant), the Gas Hourly Space Velocity within any reactor can be calculated.

In a further preferred embodiment small amounts of water are added to the process in order to improve the stability of the catalyst by reducing coke formation.

In the process according to the invention, reaction product containing one or more olefins is preferably separated into at least a first olefinic product fraction and a second olefinic fraction in a separation step. In preferred subsequent recycling step at least part of the second olefinic fraction obtained in the separation step is recycled as (part of) the olefinic co-feed.

Only part of such second olefinic fraction or the whole of the second olefinic fraction may be recycled. In a preferred embodiment, the second olefinic fraction is separated into two or more further fractions and only part of the second olefinic fraction is recycled.

Such separations can be carried out by any method known to the skilled person in the art to be suitable for this purpose, for example by vapour-liquid separation (e.g. flashing), distillation, extraction, membrane separation or a combination of such methods. Preferably the separations are carried out by means of distillation.

In one embodiment, where the purpose of the process is to prepare ethene and/or propene, the olefinic reaction product (i.e. the reaction product containing one or more olefins) obtained in the process is preferably separated into at least one olefinic product fraction containing ethene and/or propene and one or more further olefinic fraction containing olefins having 4 or more carbon atoms (i.e. C₄₊ olefins), such as butenes, pentenes, hexenes and heptenes, which further olefinic fraction is at least partly recycled.

In another embodiment, where the purpose of the process is to prepare pentenes and/or hexenes, the olefinic reaction product is preferably separated into at least one olefinic product fraction containing pentenes and/or hexenes and one or more further olefinic fractions containing olefins having 4 or less carbon atoms (i.e. C₄₋ olefins), such as butenes, propene and ethene, which further olefinic fraction is at least partly recycled.

In a still further embodiment, where the purpose of the process is to prepare ethene, propene, pentene and/or hexene, the olefinic reaction product is preferably separated into a first olefinic product fraction containing ethene and/or propene, a second olefinic product fraction containing pentenes and/or hexenes and a third olefinic fraction containing only olefins having 4 carbon atoms, which third olefinic fraction is at least partly recycled.

### EXAMPLE

In this example 1-butene and methanol were reacted in molar feed ratios butene:methanol of 1:1 and 1:2 over an MTT-type zeolite having a silica-to-alumina ratio of 47 at 2 different space velocities. All Gas Hourly Space Velocities are measured at standard temperature and pressure (STP), i.e. at 23° C. and 1 bar. As a comparison the reaction of pure 1-butene (no methanol added) has been included. A sample of zeolite powder was pressed into tablets and the tablets were broken into pieces and sieved. For catalytic testing, the sieve fraction of 30-80 mesh has been used. A quartz reactor tube of 3 mm internal diameter was loaded with 100 or 200 mg of sieve fraction. Prior to reaction, the fresh catalyst in its ammonium-form was treated with flowing argon at 550° C. for 1 hour. Next, the catalyst was cooled in argon to the reaction temperature and a mixture consisting of 2.0 vol. % 1-butene, respectively 2.0 and 3.9 vol. % methanol and 1 vol. % of water in argon was passed over the catalyst at atmospheric pressure (1 bar) at a flow rate of 50 ml/minute. Periodically, the effluent from the reactor was analyzed by gas chromatography (GC) to determine the product composition. The composition has been calculated on a weight basis. The selectivity has been defined by the division of the mass of product i by the sum of the masses of all products. The following tables (table 1) lists reaction parameters together with the compositional data, as determined by GC:

TABLE 1

| | Gas Hourly Space velocity *1000 | | | | |
|---|---|---|---|---|---|
| | (ml/gram/hr − 1) | | | | |
| | 15 | 15 | 15 | 30 | 30 |
| Methanol:1-butene | 0 | 1 | 2 | 1 | 2 |
| Temperature ° C. | 525° C. | 525° C. | 525° C. | 525° C. | 525° C. |
| 1-butene conversion, % | ~5 | 61 | 63 | 63 | 71 |
| Methanol conversion, % | — | ~100 | ~100 | ~100 | 92 |
| Ethylene, wt. %/ | ~1/ | 12/ | 8/ | 5/ | 2/ |
| selectivity, % | — | 17 | 10 | 7 | 3 |
| Propylene, wt. %/ | ~2/ | 35/ | 37/ | 21/ | 10/ |
| selectivity, % | — | 49 | 47 | 30 | 14 |
| Pentene isomers, wt. %/ | ~2/ | 20/ | 28/ | 38/ | 40/ |
| selectivity, % | — | 28 | 36 | 55 | 54 |
| Hexene isomers, wt. %/ | <1/ | 4/ | 5/ | 5/ | 22/ |
| selectivity, % | — | 6 | 6 | 7 | 30 |

As can be seen a the selectivity towards higher or lower olefins may be directed by the gas velocity. With the process according to the invention a high selectivity towards ethylene and/or propylene can be obtained. By using slightly different GHSV's a high selectivity towards pentenes and/or hexenes can be obtained.

1. A process for the preparation of an olefin comprising
contacting a diluted olefinic hydrocarbon feed, containing an oxygenate feed, an olefinic co-feed and one or more diluents, with a solid zeolite catalyst, wherein the zeolite is a one-dimensional zeolite having 10-membered ring channels, to obtain a reaction product containing one or more olefins, and wherein the diluted feed is contacted with the solid zeolite catalyst at a Gas Hourly Space Velocity, as measured at standard temperature and pressure of 23° C. and 1 bar, of at least between 10,000 to 25,000 ml diluted olefinic hydrocarbon feed/gram zeolite catalyst/hour, to prepare ethylene and/or propylene.

2. The process for the preparation of an olefin comprising
contacting a diluted olefinic hydrocarbon feed, containing an oxygenate feed, an olefinic co-feed and one or more diluents, with a solid zeolite catalyst, wherein the zeolite is a one-dimensional zeolite having 10-membered ring channels, to obtain a reaction product containing one or more olefins, and wherein the diluted feed is contacted with the solid zeolite catalyst at a Gas Hourly Space Velocity, as measured at standard temperature and pressure of 23° C. and 1 bar, of between 30,000 to 300,000 ml diluted olefinic hydrocarbon feed/gram zeolite catalyst/hour, to prepare pentenes and/or hexenes.

3. The process according to claim 1, wherein the oxygenate feed contains methanol and/or dimethylether.

4. The process according to claim 1, wherein the zeolite is a zeolite of the MTT-type or the TON-type.

5. The process according to claim 1, wherein at least part of the olefins in the obtained reaction product are recycled as an olefinic co-feed.

6. The process according to claim 1, wherein the process is carried out at a temperature in the range from 450° C. to 550° C.

7. The process according to claim 1, wherein the ratio of mol oxygenate to mol olefin lies in the range of 10:1 to 1:10.

8. The process according to claim 1, wherein a reaction product containing one or more olefins is separated into a first olefinic product fraction and a second olefinic fraction in a separation step.

9. The process according to claim 8, wherein part of or the whole of the second olefinic fraction is recycled to form part or all of the olefinic co-feed.

10. The process according to claim 1, wherein the olefinic co-feed contains olefins having 4 or more carbon atoms.

11. The process according to claim 2, wherein the olefinic co-feed contains olefins having 4 or less carbon atoms.

12. The process according to claim 1, wherein the olefinic co-feed contains only olefins having 4 carbon atoms.

13. The process according to claim 2, wherein the oxygenate feed contains methanol and/or dimethylether.

14. The process according to claim 2, wherein the zeolite is a zeolite of the MTT-type or the TON-type.

15. The process according to claim 2, wherein at least part of the olefins in the obtained reaction product are recycled as an olefinic co-feed.

16. The process according to claim 2, wherein the process is carried out at a temperature in the range from 450° C. to 550° C.

17. The process according to claim 2, wherein the ratio of mol oxygenate to mol olefin lies in the range of 10:1 to 1:10.

18. The process according to claim 2, wherein a reaction product containing one or more olefins is separated into a first olefinic product fraction and a second olefinic fraction in a separation step.
